(19) United States
(12) Patent Application Publication
(10) Pub. No.: US 2022/0378123 A1
(43) Pub. Date: Dec. 1, 2022

(54) TEMPERATURE CONTROLLED SYSTEM FOR RECONSTRUCTED AND AUGMENTED BREASTS

(71) Applicants: Carolina Rojas, Los Angeles, CA (US); Dominique Aris, Rolling Hills Estates, CA (US)

(72) Inventors: Carolina Rojas, Los Angeles, CA (US); Dominique Aris, Rolling Hills Estates, CA (US)

(21) Appl. No.: 17/826,531

(22) Filed: May 27, 2022

Related U.S. Application Data
(60) Provisional application No. 63/193,743, filed on May 27, 2021.

(51) Int. Cl. A41D 13/005; A61F 7/02

(52) U.S. Cl. CPC A41D 13/0051; A61F 7/02; A41C 3/0064

(57) ABSTRACT

A temperature controlled garment or bra system can be integral with a bra or a separate thermal insert that can be coupled to any type of garment. The thermal insert having one or more thermal zones or regions that are configured to form a temperature gradient for different breast regions for the purpose of having a uniform breast implant temperature.

### PRIORITY

This application claims priority to and the benefit of U.S. Provisional Patent Application No. 63/193,743 filed on May 27, 2021, which is incorporated herein by reference in its entirety.

### FIELD

The present invention relates generally to temperature controlled systems, devices, and methods that can be used alone or in conjunction with various garments and more particularly, to a temperature controlled system that can be used alone or with a brassieres to warm the breast implants and surrounding tissue of augmented or reconstructed breasts.

### BACKGROUND

Temperature controlled garments or apparel have been developed to keep various parts of a person's body warm or cooled. These garments have typically focused on keeping a person's extremities (e.g., feet and hands) warm during cold seasons. Other temperature controlled devices have been developed to keep a person cool. These devices generally include hats and devices placed around a person's neck. Similarly, temperature controlled articles (e.g., patches) have been developed to produce warm or cool temperatures to a particular part of a person's body for the purpose of treating an injury.

Attempts have been made to develop garments or brassieres that can be temperature controlled. An ability to control the temperature of a user's chest is particularly important to women that have breast implants inserted into their chests during breast reconstruction or augmentation surgery. It is generally known that after implantation, breast implants tend to make a women's breasts and/or chest cold. The coldness experienced by women is particularly acute in those that have lost significant breast tissue as a result of undergoing breast reconstruction surgery. For all breast implants, but especially those used in breast reconstruction procedures, the cool or cold sensation is a result of a reduction or loss of blood flow through the breast tissue, which normally keeps the breast and chest area close to normal body temperature. For breast reconstruction patients, however, the blood flow is limited to the skin covering the breast implant. As a result, the breast implant and surrounding tissue typically have a temperature approximately equal to the ambient temperature of the patient's surroundings, which can have a delta or difference of 20 or more degrees Fahrenheit from normal body temperature. It is also believed that a breast implant acts as a barrier to core body temperature reaching the distal portion or nipple area of a breast. As a result, it is believed that a temperature gradient is formed with a temperature of breast tissue decreasing from the chest wall toward the nipple.

Conventional temperature controlled brassieres generally operate by either inserting a non-electrical object, such as silicone pack that can be heated by being placed in a microwave or cooled by being placed in a freezer, into a pocked formed in the brassieres. These passive thermal control devices have no temperature gradient so as women's breast closest to the chest is able to be too warm while breast tissue near the nipple may not be warm enough. Additionally, these conventional packs typically start either very hot or very cold and lose their temperature regulating properties very quickly. Additionally, they tend be rather large, bulky, and rigid. As a result, the breast do not look natural while the passive devices are being worn.

To overcome the problems with the passive devices, electrical devices were developed in an attempt to provide more uniform temperature control with less weight. However, these conventional temperature brassieres require large power supplies and large heating elements that have proven to be commercially unsatisfactory.

Conventional temperature controlled brassieres have also largely been sewn into the interior of conventional brassieres. As a result, extra care has to be taken when washing or cleaning such a brassiere or bra in order to avoid damaging the electrical components. In today's hectic life, people strive to find ways to simplify tasks such as laundry. With such a strain on time, conventional temperature controlled brassieres or bras have failed to garner consumer interest.

In all of the devices available or described a need has remained for an improved temperature controlled brassieres or bra that is able to selectively control the heating and cooling of select parts a body part or areas, such as a breast having a breast implant. There is also a need for a temperature controlled brassiere or bra that is able to create a temperature gradient to allow for a uniform temperature across an augmented or reconstructed breast. There is also a need for a temperature controlled system that can be used without a bra or with other tops and materials in order to reduce costs, increase compliance, and allow women to maintain a uniform breast temperature while engaged in activities such as swimming. There is also still a need for a temperature controlled garment that is lightweight, can be used with any unconventional brassieres or clothes, is easily cleaned, and doesn't alter the contour of the surface of the breast.

### SUMMARY

The device and assemblies described herein provide a temperature controlled system, device, and garments, such as brassieres and other tops, that can be controlled to selectively heat and/or cool select areas of a body part, such as a breast having a breast implant. The device and assemblies are also lightweight and can be used with any conventional brassieres. The system, device and assemblies of the present invention are easily cleaned and do not alter the contour of the surface of the breast when worn.

It is a feature of the present invention to provide a temperature controlled system, device and garment that is lightweight and conforms to the body part being heated or cooled.

It is another feature of the present invention to provide a temperature controlled system, device and garment that is can be discreetly controlled by a remote device or operation that mimics normal garment adjustment features.

It is still another feature of the present invention to provide a temperature controlled system, device and garment that is easily assembled and disassembled to enable increased use and easy cleaning.

Further objects, features and advantages of the invention will be apparent from the following detailed description when taken in conjunction with the accompanying drawings.

The above summary is not intended to limit the scope of the invention, or describe each embodiment, aspect, implementation, feature or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1A is a front view of a unitary temperature controlled garment according to an example embodiment.

FIG. 1B is a front perspective view of a unitary temperature controlled garment according to an example embodiment.

FIG. 1C is a side view of a unitary temperature controlled garment according to an example embodiment.

FIG. 2 is a front view of an on-board controller of a unitary temperature controlled garment according to an example embodiment of the invention.

FIG. 3A is a perspective view of a power supply holder of a unitary temperature controlled garment cover according to an example embodiment.

FIG. 3B is a front view of a power supply holder of a unitary temperature controlled garment cover according to an example embodiment.

FIG. 4 is a close up perspective view of a heating element of a temperature controlled garment according to an example embodiment.

FIG. 5 is a front view of a cup member of a temperature controlled garment according to an example embodiment.

FIG. 6A is a perspective view of a power control assembly of a temperature controlled garment according to an example embodiment.

FIG. 6B is a perspective view of a mobile phone and smart watch control assembly according to an example embodiment.

FIG. 7 is a perspective view of a temperature control element of a temperature controlled garment according to an example embodiment.

FIG. 8A is a cross section view of a temperature control element of a temperature controlled garment according to an example embodiment.

FIG. 8B is a perspective view of a temperature control element of a temperature controlled garment according to an example embodiment.

FIG. 9A is a front view of a support member used in a temperature controlled garment assembly according to an example embodiment.

FIG. 9B is a back view of a support member used in a temperature controlled garment assembly according to an example embodiment.

FIG. 10A is a front view of a temperature control element example heating pattern used in a temperature controlled garment assembly according to an example embodiment.

FIG. 10B is a front view of a temperature control element used in a temperature controlled garment assembly according to an example embodiment.

FIG. 10C is a front view of a combined support member and temperature controlled element of a temperature control garment assembly according to an example embodiment.

FIG. 11A is a front view of a support member used in a temperature controlled garment assembly according to an example embodiment.

FIG. 11B is a back view of a support member used in a temperature controlled garment assembly according to an example embodiment.

FIG. 12A is a front view of a temperature control element example heating pattern used in a temperature controlled garment assembly according to an example embodiment.

FIG. 12B is a front view of a temperature control element used in a temperature controlled garment assembly according to an example embodiment.

FIG. 12C is a front view of a combined support member and temperature controlled element of a temperature control garment assembly according to an example embodiment.

FIG. 13A is a front view of a support member used in a temperature controlled garment assembly according to an example embodiment.

FIG. 13B is a back view of a support member used in a temperature controlled garment assembly according to an example embodiment.

FIG. 14A is a front view of a temperature control element example heating pattern used in a temperature controlled garment assembly according to an example embodiment.

FIG. 14B is a front view of a temperature control element used in a temperature controlled garment assembly according to an example embodiment.

FIG. 14C is a front view of a combined support member and temperature controlled element of a temperature control garment assembly according to an example embodiment.

FIG. 15A is a front view of a support member used in a temperature controlled garment assembly according to an example embodiment.

FIG. 15B is a front view of a temperature control element used in a temperature controlled garment assembly according to an example embodiment.

FIGS. 15C and 15D are front views of a combined support member and temperature control being coupled together according to an example embodiment.

FIG. 16A is a front view of a support member used in a temperature controlled garment assembly according to an example embodiment.

FIG. 16B is a front view of a temperature control element used in a temperature controlled garment assembly according to an example embodiment.

FIGS. 16C and 16D are front views of a combined support member and temperature control being coupled together according to an example embodiment.

FIG. 17A is a front view of a support member used in a temperature controlled garment assembly according to an example embodiment.

FIG. 17B is a front view of a temperature control element used in a temperature controlled garment assembly according to an example embodiment.

FIGS. 17C and 17D are front views of a combined support member and temperature control being coupled together according to an example embodiment.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION

In the following descriptions, the present invention will be explained with reference to various exemplary embodiments. Nevertheless, these embodiments are not intended to limit the present invention to any specific example, environment, application, or particular implementation described herein. Therefore, descriptions of these example embodiments are only provided for purpose of illustration rather than to limit the present invention.

Dimensions and relative proportions of components are merely example embodiments and can be varied unless specifically limited in a given claim. Thus, the dimensions can be varied without departing from the scope of the invention.

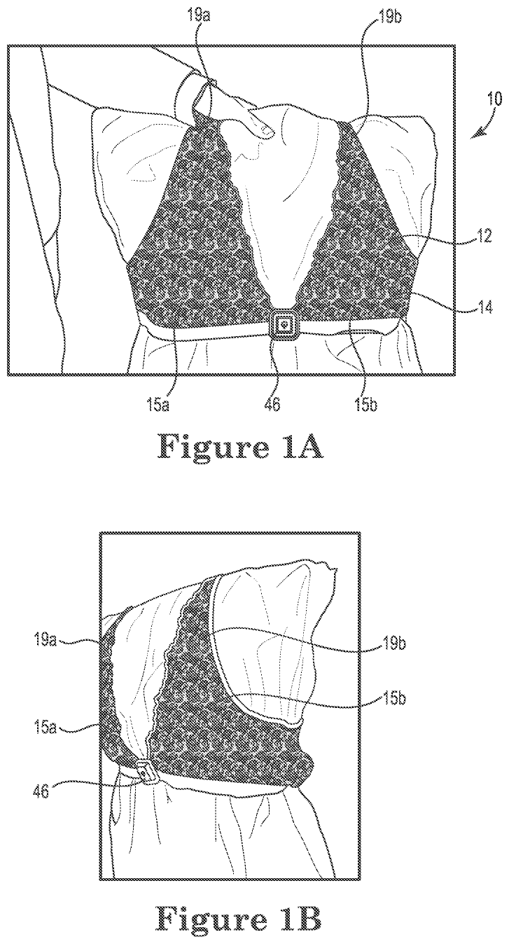

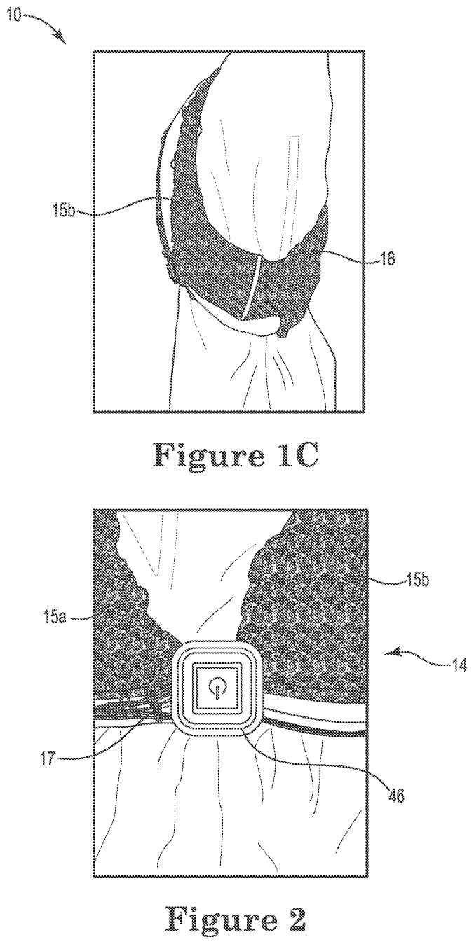

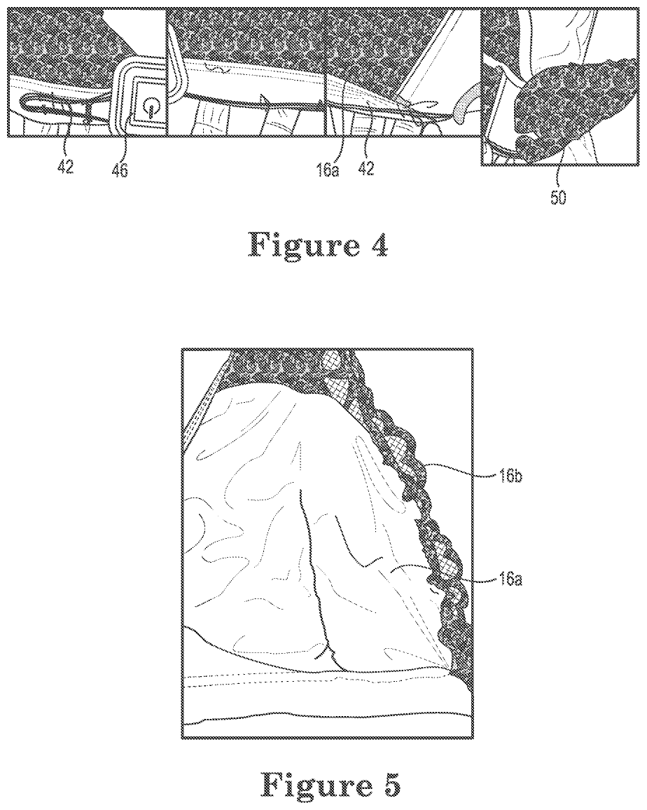

As illustrated in FIGS. 1-17D, the present invention of a temperature controlled garment system 10 comprises one or more articles of clothing 12 and one or more temperature control elements 20 operatively coupled together and capable of being worn by a person or animal, such as horses, dogs, cats, and the like. The temperature control assembly 40 is configured to be selectively controlled by a user to heat or cool a particular part of the body covered by at least a portion of the article of clothing 12.

Temperature controlled clothing is particularly important to users that have had devices implanted into their bodies that cause a change in their body or impacted tissue to self-regulate a temperature. As a result, the impact tissue or body part tends to feel cool or cold to the touch. This is particularly relevant to women that have undergone breast reconstruction or augmentation that includes the implanting of a breast implant.

Referring to FIGS. 1A and 1B, in one example embodiment of the present invention the article of clothing 12 can comprises a brassier or bra 14 designed to be worn to warm, or optionally cool, the breast tissue and/or breast implant of a user. The bra 14 includes a first cup 15a and a second cup 15b that are each configured to receive a breast of a user. The temperature controlled element 40 is in operative communication with at least one of the first cup 15a or the second cup 15b. Since some users may only have one breast augmented or reconstructed, the present invention does not require both bra cups to have a temperature controlled assembly 40.

In one configuration of the present invention, the first cup 15a and the second cup 15b can include an inner surface 16a and an outer surface 16. Additionally, the first cup 15a and the second cup 15b can be configured constructed from multiple layers of material such that one or more pockets are formed that are adapted to hold one or more temperature control elements 20. The temperature control element 20 can be placed against the inner surface 16a, the outer surface 16b, or positioned in one or more of the pockets.

As particularly illustrated in FIGS. 1A and 2, the bra 14 can include a bridge 17 that is coupled to and extends between the first cup 15a and the second cup 15b. A back strap or band 18 can be coupled to and extend between the first cup 15a and the second cup 15b and configured to extend between each of the cups 15a and 15b and around a back of a user. The back strap 18 can be manufactured to have a latching mechanism that separates the back strap 18 into back strap portions that enables the bra 14 to be more easily worn and removed.

Referring back to FIGS. 1A and 1B, a first shoulder strap 19a and a second shoulder strap 19b can be coupled to and extend between the first cup 15a, second cup 15b, and the back strap 18. The first and second shoulder straps 19a and 19b are configured to extend over the shoulders of a user. The shoulder straps 19a and 19b can be manufactured with one or more slider adjusters or rings 20 that are configured to adjust a length of the shoulder straps 19a and 19b.

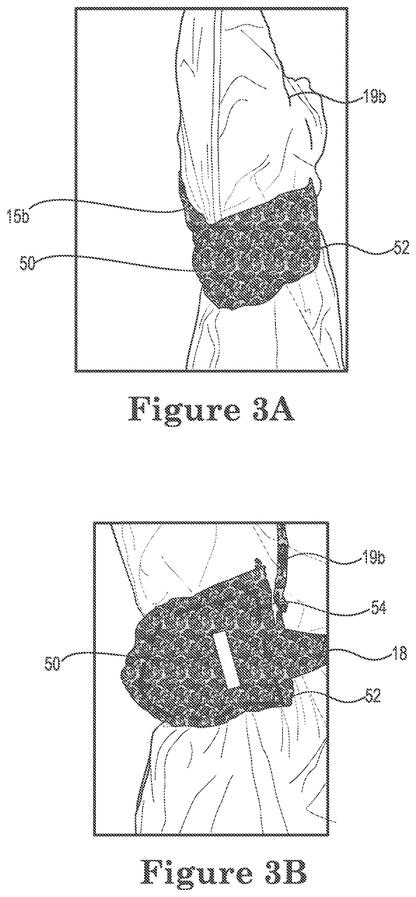

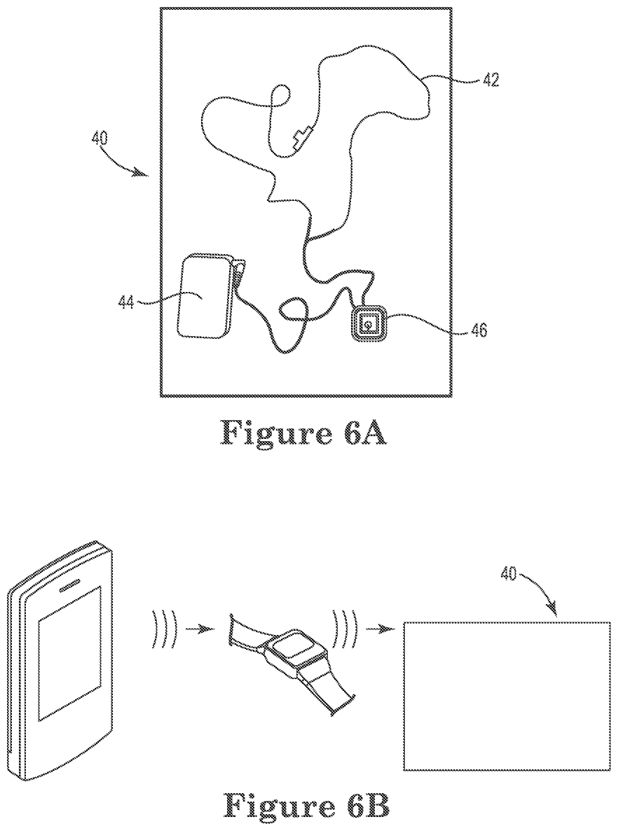

FIGS. 3A, 3B, and 6, illustrate a temperature control assembly 40 that can be incorporated into the bra 14. The temperature control assembly 40 comprises one or more temperature elements 42 coupled to a power supply 44 and a controller 46. The temperature elements 42 are configured to be coupled the first cup 15a, second cup 15b, or both. In one example embodiment, the temperature elements 42 comprise one or more wires configured to be heated or cooled by various methods, including but not limited to passing an electric current through the wire or having the wire connected to a heat sink that draws heat out of the wire.

As illustrated in FIGS. 1A, 2, and 6, the controller 46 is configured to be coupled to a portion of the bra 14 to be easily accessible to the wearer. In one example embodiment, the controller 46 is coupled to or formed as part of the bridge 17. In another example embodiment, the controller 46 is coupled to or formed as part of one or both shoulder straps 19a and 19b. In this configuration a wearer is able to actuate the controller in a discreet manner as if adjusting the bra 14.

The controller 46 comprises an actuator that is configured to adjust various features of the present invention. In one example embodiment, the controller 46 controls a temperature, properties of one or more zones, and an on and off state of the system 10. The controller 46 can be manufactured as one or more push buttons, slider switch, touch sensor, and the like. The controller 46 can also include a temperature sensor that can detect the temperature of one or more heating or cooling elements of the garment. The temperature sensor by itself or in conjunction with the controller 46, can control the device. The controls can include, but are not limited to automatically power off the device, reduce or increase a temperature, cycle between on and off states, and/or notify the user.

Referring to FIGS. 3A and 3B, a holder 50 configured to hold the power supply 44 is coupled to or formed as part of the bra 14. The holder 50 can be manufactured as a pouch having an opening 52 to an interior 54 (defined by white dashes) that is configured to receive and hold the power supply 44. The holder 50 is coupled to the bra 14 by stitching or a clipping mechanism. As particularly illustrated in FIGS. 3A and 3B, the holder 50 is secured to a portion of the back strap 18 and positionable on the side or back of the user.

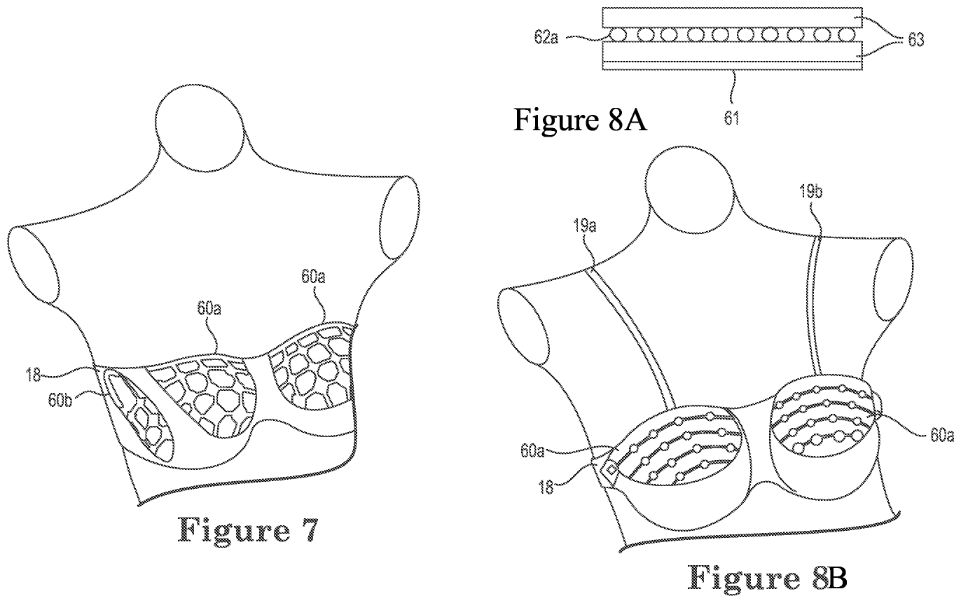

Referring to FIGS. 7, 8A, and 8B, the temperature control element 42 comprises one or more zone members 60a and 60b that are configured to operate alone or together to enable customized heating areas. The one or more zone members 60a and 60b include one or more temperature transmission members or regions 62a that are interconnected by one or more coupling mechanisms such as a wire. The temperature transmission members or regions 62a are configured to have varying properties such as different surface areas, thicknesses, resistances, and the like. The zone members 60a and 60b are configured to be controlled by a remote device (such as a cellphone, tablet, computer, or smart watch) or the controller 46. In another embodiment, one or more parts of the system 10, including the zone members 60a and 60b may be controlled by voice control.

The zone members 60a and 60b are positionable between a breast and bra 14; coupled to the inner surface 16a of the bra 14; and/or positioned in a pocket of the bra 14. As illustrated in FIGS. 7, 8A, and 8B, the zone members 60a and 60b are configured to be positioned on the superior, inferior, medial or lateral surfaces of a breast. The zone members 60a and 60b are also capable of covering a distal surface of a breast. A zone member covering a distal aspect of a breast may have an opening, hole, void, channel or carve-out formed therein in order to avoid covering a nipple of a breast.

In one embodiment of the present invention, the zone members 60a and 60b are manufactured with at least one of its surfaces having a releaseable adhesive 61 to enable a wearer to place the zone members 60a and 60b against the breast area. The adhesive 61 permits the zone members 60a and 60b to be adjustably positioned on the breast and between the breast and any outer garment such as a bra 14, shirt, chemises, corsets, bustiers, and camisoles. The adhesive 61 also enables the system 10 to be used with other types of clothing. For instance, some user wear clothing made from latex, rubber, and leather. These types of clothing tend not to lend themselves to having a temperature controlled system such as the present invention to be coupled to it. As such, the adhesive 61 enables control of a temperature of a body part such as a breast without the need to modify the clothing.

In one example embodiment, the zone members 60a and 60b may be at least partially enclosed, encased in, or sandwiched between one or more layers that are generally flexible to permit contouring when placed against a breast. The layers may have the same or different properties. For instance, an outer non-breasting contacting layer 63a may be manufactured from a soft silicone or silicone like material that mimics the feel of breast tissue. An inner layer 63b closer to the breast tissue may have thermal transferring properties to enable more efficient heating and cooling of the breast tissue. The flexible member 63 may a breast facing surface adhesive. The layers 63a and 63b can completely enclose the temperature control assembly 40 or just its elements 42 to enable the present invention to be waterproof and thereby worn during swimming and/or worn with swimwear. Any material providing these advantages may be used.

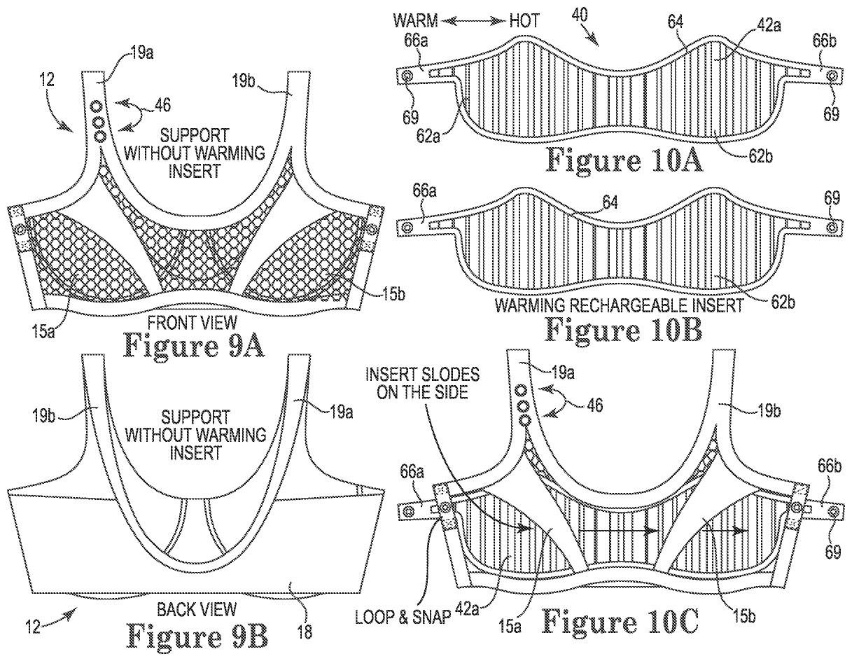

FIGS. 9A-17A are examples of various temperature controlled garment systems and assemblies 10. FIGS. 9A-10c illustrate a temperature control assembly 40 having one or more a temperature transmission members or elements 42 that can be divided into one temperature transmission regions or zones 62a and 62b extending in a vertical direction between a superior and inferior aspect of the wearer. The temperature transmission regions or zones 62a and 62b are configured to increase in surface area from lateral and medial aspects toward the nipple. This configuration enables a temperature gradient that increases from the lateral and medial aspects of the breast toward the nipple. This type of configuration is generally ideal as breast tissue furthest from the torso generally has less tissue and blood supply, and is therefore generally cooler. This configuration provides a varying laterally and medially distributed temperature gradient. It also provides a consistent superior to inferior temperature gradient.

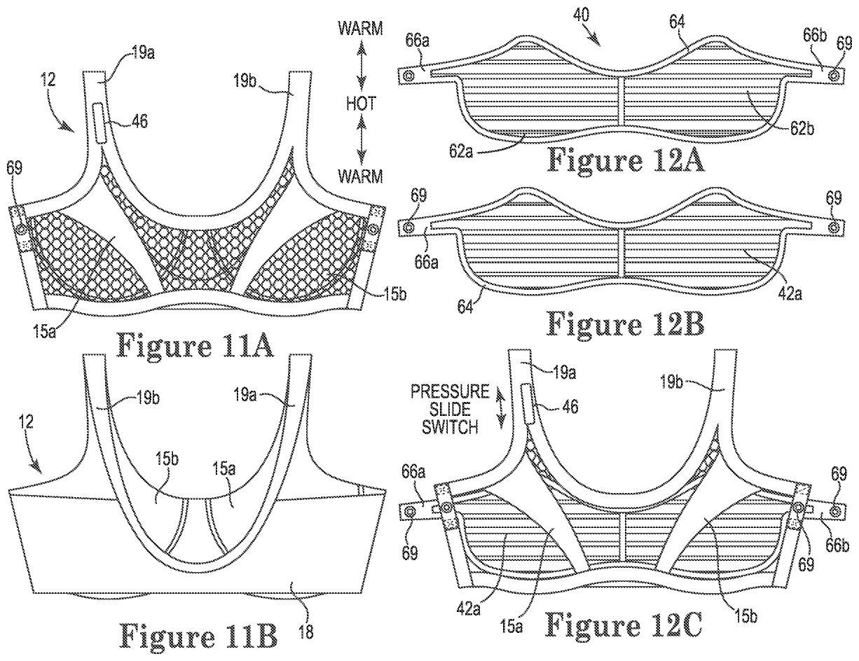

FIGS. 11A-12C illustrate a temperature control assembly 40 having one or more temperature transmission elements 42 with temperature transmission regions or zones 62a and 62b extending in a horizontal direction between a lateral and medial aspect of the wearer. The temperature transmission regions or zones 62a and 62b are configured to increase in surface area from superior and inferior aspects of the wearer towards their nipple. This configuration enables a temperature gradient that increases from the superior and inferior aspects of the breast toward the nipple. This configuration provides a superior and inferior distributed temperature gradient with a generally consistent laterally to medially temperature gradient.

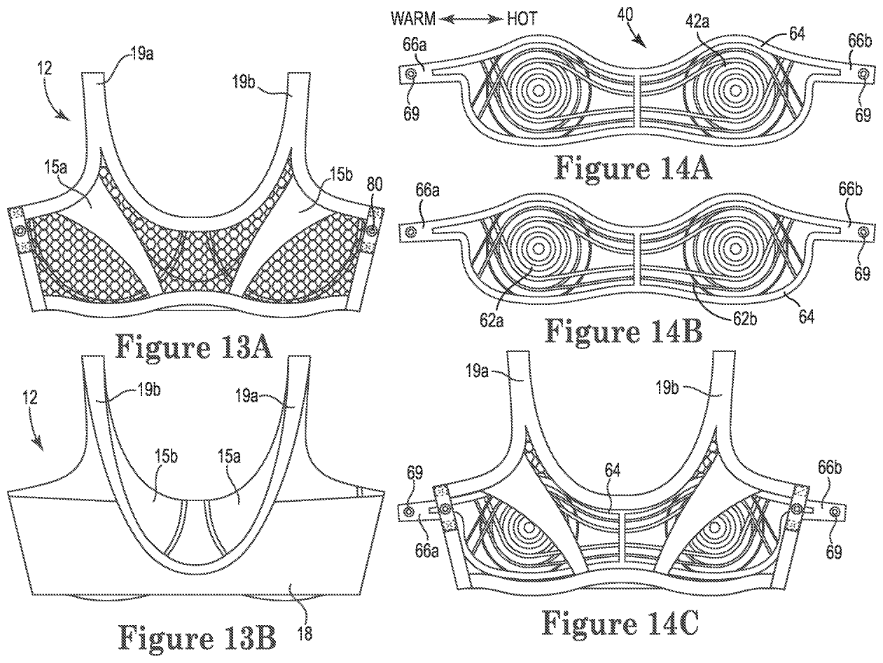

FIGS. 13A-14C illustrate a temperature control assembly 40 having one or more temperature transmission elements 42 with one or more temperature transmission regions or zones 62a and 62b extending in generally concentric configuration with members nearer a nipple of a user having a greater surface area than members further away a user's nipple. This configuration enables a temperature gradient that increases from a user's torso toward the user's nipple. This configuration provides concentrically distributed temperature gradient with to provide more warmth or cooling of breast tissue further from the body.

As particularly illustrated in FIGS. 9A-14C, the temperature transmission element 42 comprises an edge or trim member 64 that extends at least partially about its perimeter edge. While not required, the trim member 64 can also be temperature controlled by the controller 46. The temperature transmission element 42 may also comprise one or more laterally extending arm portions 66a and 66b that are configured to be coupled to a portion of the garment or bra 14. In this example embodiment, one or more fasteners 69 (such as a snap faster or hook and loop fasteners) are placed on ends of the arm portions 66a and 66b and are able to be releasably fastened to a corresponding fastener on the garment or bra 14 or back onto the temperature control element 42.

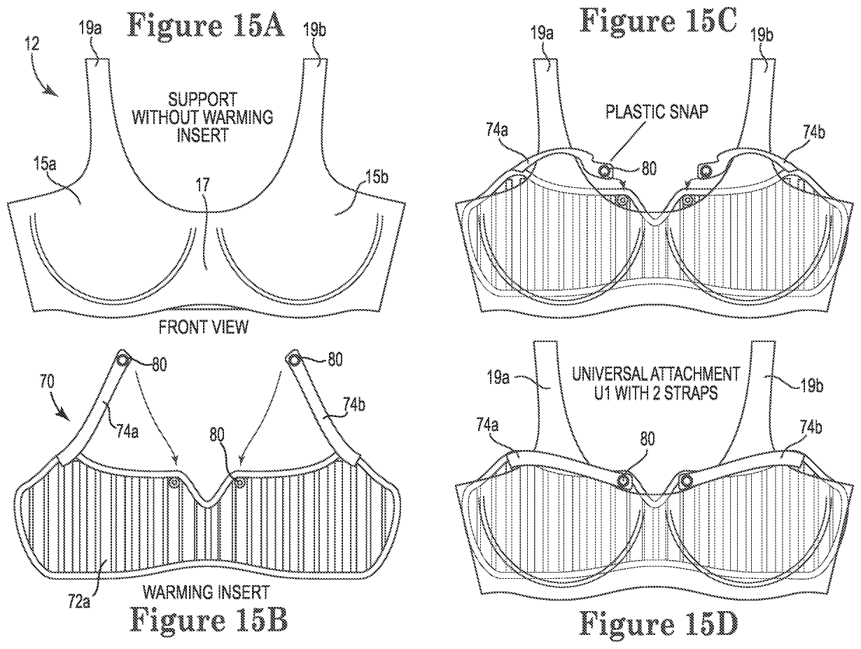

FIGS. 15A-17D illustrate additional embodiments of the present invention. Each of these embodiments comprise a temperature control assembly 40 that includes a garment or bra 12 and a temperature transmission element or insert 70 having temperature transmission regions or zones 72a and 72b extending in a vertical direction between a superior and inferior aspect of the wearer. The temperature transmission regions or zones 72a and 72b are configured to increase in surface area from lateral and medial aspects toward the nipple. This configuration enables a temperature gradient that increases from the lateral and medial aspects of the breast toward the nipple. This type of configuration is generally ideal as breast tissue furthest from the torso generally has less tissue and blood supply, and is therefore generally cooler. This configuration provides a varying laterally and medially distributed temperature gradient. It also provides a consistent superior to inferior temperature gradient. The embodiments of FIGS. 15A-17D can have any configuration of temperature transmission regions or zones 72a and 72b disclosed herein.

The temperature transmission element or insert 70 includes one or more arms 74a, 74b, 74c and 74d coupled to and extending from a peripheral or perimeter trim, edge or frame portion 76 of the heating element 70. The arms 74a, 74b, 74c and 74d are configured to extend around a portion of the garment or bra 14 and to either detachably fasten to the garment or bra 14, or to itself such that it remains positioned between the breast and the garment 12.

Referring to FIGS. 15A-15D, the temperature transmission element or insert 70 includes arms 74a and 74b extending in an upwardly direction and are configured to extend about shoulder straps 19a and 19b. One or more fasteners 80 are disposed on a portion of arms 74a and 74b and a portion of the temperature transmission element or insert 70, whereby the arms 74a and 74b are selectively secured to the garment or bra 12. Any fastener 80 may be used, including but not limited to a snap faster, hook and loop fasteners, magnets, and the like.

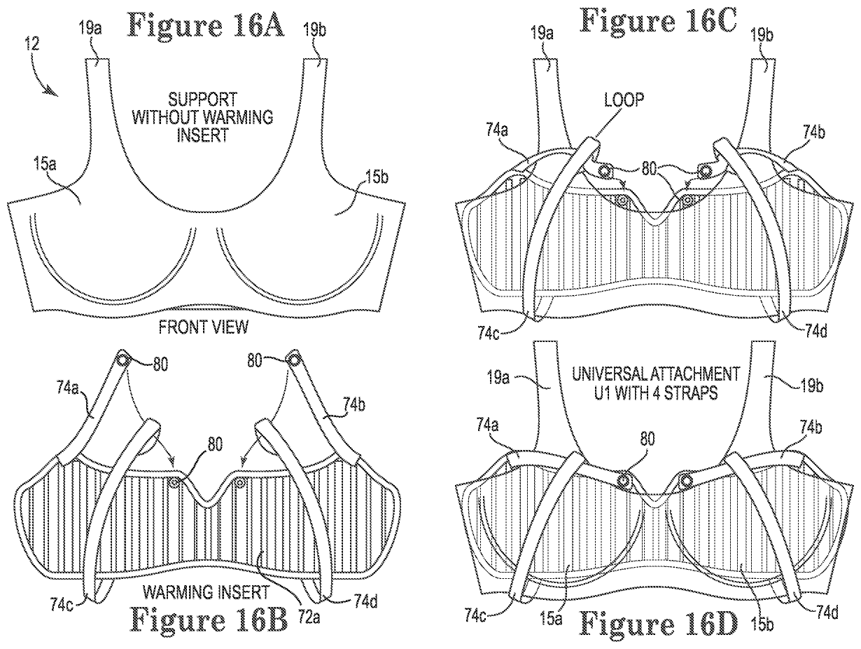

Referring now to FIGS. 16A-16D, the temperature transmission element or insert 70 includes arms 74a, 74b, 74c and 74d configured to extend about the garment or bra 14. Arms 74a and 74b are configured to extend in an upwardly direction and are configured to extend about shoulder straps 19a and 19b. Arms 74c and 74d are configured to extend in a downwardly direction and then fold over the first cup 15a and the second cup 15b. One or more fasteners 80 are disposed on a portion of arms 74a and 74b, and a portion of the temperature transmission element or insert 70, whereby the arms 74a and 74b are selectively secured to the garment or bra 12. Fasteners 80 disposed on a portion of arms 74c and 74d, and another portion of arms 74c and 74d. Once arms 74a and 74d are secured, arms 74c and 74d are positioned over arms 74a and 74b when secured and are selectively secured to themselves by fastener 80. It is also contemplated herein that arms 74c and 74d may be fastened to a portion of the heating element 70 or to arms 74a and 74d. Any fastener 80 may be used, including but not limited to a snap faster, hook and loop fasteners, magnets, and the like.

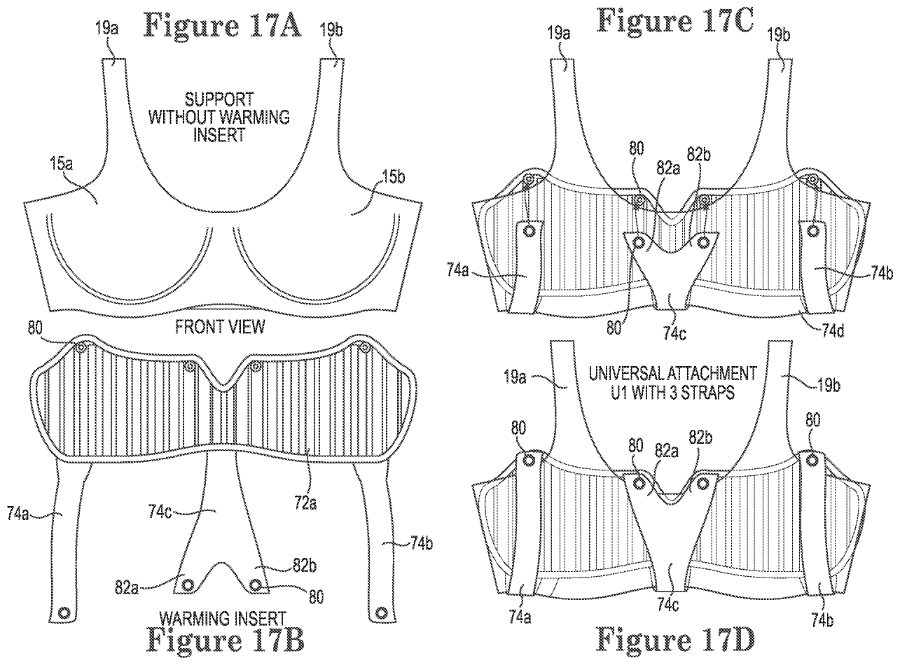

Referring now to FIGS. 17A-17D, the temperature transmission element or insert 70 includes arms 74a, 74b, and 74c configured to extend about the garment or bra 14. Arms 74a and 74b are configured to extend in a downwardly direction and are configured to be folded up and extend about lateral sides of the first cup 15a and the lateral side of second cup 15b whereby they are fastened to a portion of the temperature transmission element or insert 70. Arm 74c extends in a downwardly direction from the temperature transmission element or insert 70 and is configured to be folded over the bridge 17 whereby it can be selectively fastened to a portion of the temperature transmission element or insert 70. Arm 74c comprises one or more extension portions 82a and 82b that extend in a generally Y configuration from an end of arm 74c. The Y configuration enables arm 74c to have a shape that conforms to the shape of the breasts. One or more fasteners 80 are disposed on a portion of arms 74a, 74b, and 74c (or extensions 82a and 82b) and portions of the temperature transmission element or insert 70, whereby the arms 74a, 74b, and 74c are selectively secured to the garment or bra 12.

It is within the spirit and scope of this invention that arms 74a, 74b, 74c, 74d, or extensions thereof may be fastened to a portion of the temperature transmission element or insert 70, to other arms, or to themselves. As stated above, any fastener 80 may be used, including but not limited to a snap faster, hook and loop fasteners, magnets, and the like.

The device of the present invention is capable of using any material that is able to transfer or emit hot, warm, cool, and cold temperatures. The material can comprise any number of cells that can be controlled by the controller 46. The controller 46 is able to control individual cells, grounds or zones of cells, or all cells. The cells are able to have similar or distinct temperatures and ranges to allow a user to control the temperature of various parts of their body. The cells can all have the same or different shapes. Additionally, the shape of the cells can one or more shapes that conform to a shape of the garment being used.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A temperature controlled garment assembly for selectively warming breasts with or without nipples, the breasts containing breast implants, the assembly comprising:
a breast garment comprising a first cup portion and a second cup portion, the cup portions being designed to be disposed about a portion of the breasts, each cup portion comprising an inner panel member and an outer panel member defining an interior, the inner panel member of each cup portion being configured to be disposed adjacent to the breasts;
a temperature control element configured to emit heat being disposed in the interior of at least one of the cup portions;
a controller comprising a power supply and an actuator operatively coupled to the temperature control element to control the temperature emitted, the controller being disposed on an easily accessible portion of the breast garment; and
wherein the temperature control element is configured to emits heat to warm the breast tissue containing the breast implant.

2. The temperature controlled garment assembly of claim 1, wherein each of the cup portions contain a temperature control element that are separately controllable by the controller.

3. The temperature controlled garment assembly of claim 1, wherein the temperature control element comprises a plurality of zones, with each of the plurality of zones being separately controllable by the controller.

4. The temperature controlled garment assembly of claim 1, wherein the breast garment comprises an opening extending through the inner panel and the outer panel configured to permit passage of the nipple of the breasts to prevent their overheating.

5. The temperature controlled garment assembly of claim 1, further comprising a heatsink disposed on the breast garment and operatively coupled to the temperature control element, wherein the heatsink is configured to draw heat from the breast through the temperature control element.

6. The temperature controlled garment assembly of claim 5, further comprising a bridge member being coupled to and extending between the cup portions, wherein the heatsink is disposed on the bridge member to cool a location between the breasts.

7. An augmented breast temperature regulator device for selectively warming breasts with or without nipples containing breast implants, the device being removably coupled to a breast garment having first and second breast cups, the device comprising:
a temperature regulator comprising a flexible substrate and at least one temperature control element coupled to and extending along a portion of the flexible substrate, the temperature control element is configured to emit heat;
a controller comprising a power supply and an actuator operatively coupled to the temperature control element to control a temperature of the temperature control element; and
wherein the controller is removably coupled to a portion of the flexible substrate.

8. The augmented breast temperature regulator of claim 7, wherein the flexible substrate further comprises one or more elongate arms that are extendable about a portion of the breast garment to reasonably couple the temperature regulator to the breast garment.

9. The augmented breast temperature regulator of claim 8, further comprising one or more fasteners attached to the one or more elongate arms and portions of the flexible substrate, wherein the one or more fasteners releasably fasten the one or more elongate arms to the flexible substrate.

10. The augmented breast temperature regulator of claim, 8 wherein the one or more elongate arms comprise one or more downwardly extending arms and one or more upwardly extending arms, wherein the controller is removably coupled to one of the upwardly extending arms.

11. The augmented breast temperature regulator of claim 8, wherein the one or more elongate arms comprise at least one middle arm and a pair of end arms extending downwardly from the flexible substrate, wherein the controller is removably coupled to the a portion of the middle arm.

12. The augmented breast temperature regulator of claim 7, the wherein the substrate comprises a first cup portion and a second cup portion, each cup portion contains a temperature control element that are separately controllable by the controller, wherein the controller is removably coupled to the bridge portion.

13. The augmented breast temperature regulator of claim 7, wherein the temperature control element comprises a plurality of zones, with each of the plurality of zones being separately controllable by the controller.

14. The augmented breast temperature regulator of claim 12, further comprising sensors coupled to the substrate and configured to detect a temperate of the breast in the plurality of zones, wherein the controller is configured to heat the plurality of zones according to a temperature detected by the sensors.

15. The augmented breast temperature regulator of claim 14, wherein the controller heats the plurality of zones from opposed ends of the flexible substrate towards a middle region of the flexible substrate.

16. The augmented breast temperature regulator of claim 14, wherein the controller heats the plurality of zones from a lower edge of the flexible substrate towards an upper edge of the flexible substrate.

17. The augmented breast temperature regulator of claim 14, wherein the controller heats the plurality of zones radially from an outer edge of the flexible substrate towards a middle region of the flexible substrate.

18. The augmented breast temperature regulator of claim 7, further comprising a water resistant and temperature transmitting cover having an interior pocket configured to receive the temperature regulator, wherein the water resistant and temperature transmitting cover protects the temperature regulator from water.

19. A method of selectively warming breasts, with or without nipples, which have breast implants, the method comprising the steps of:
providing a breast garment comprising a first cup portion and a second cup portion;
providing a flexible temperature control element configured to emit heat;
coupling the flexible temperature control element to the breast garment;
wearing the breast garment and the flexible temperature control element, wherein the flexible temperature control element is disposed between the breasts and the breast garment;
providing a controller comprising a power supply and an actuator operatively coupled to the flexible temperature control element;
operating the controller to heat the flexible temperature control element; and
wherein the heat emitted from the flexible temperature control element is transmitted to the breast having the breast implant.

20. The method of claim 19, wherein the flexible temperature control element comprises a plurality of zones, wherein the controller is selectively controllable to heat select zones.
